(19) United States
(12) Patent Application Publication
(10) Pub. No.: US 2014/0373845 A1
(43) Pub. Date: Dec. 25, 2014

(54) METHODS AND SYSTEMS FOR ADAPTIVE ADJUSTMENT OF VENTILATOR SETTINGS

(71) Applicant: Covidien LP, Boulder, CO (US)

(72) Inventor: Nancy F. Dong, Carlsbad, CA (US)

(21) Appl. No.: 13/926,013

(22) Filed: Jun. 25, 2013

(51) Int. Cl. A61M 16/00; A61B 5/085; A61M 16/08; A61B 5/087; A61B 5/00

(52) U.S. Cl. CPC A61M 16/0051; A61M 16/0069; A61B 5/087; A61B 5/4836; A61M 16/0003; A61M 16/0883; A61B 5/085; A61M 2016/0027; A61M 2016/0039; A61M 2016/0042
USPC 128/204.23

(57) ABSTRACT

This disclosure describes systems and methods for providing optimized adjustment of a ventilator based on an estimated net value of patient effort. The disclosure describes a novel breath type that delivers a target airway pressure calculated based on an estimated patient muscle effort. A net value of patient muscle effort is estimated using a parameter estimate vector update equation to solve for a recursive least squares gain value representing the estimated net value of patient effort. The estimated net value of patient muscle effort is used to determine a target airway pressure, which is then used to determine a target inspiratory pressure to be delivered to a patient. The target inspiratory pressure is used to determine an onset and/or end of a ventilation cycle and therefore improves the synchronization between the ventilator and the patient.

Medical ventilator systems have long been used to provide ventilatory and supplemental oxygen support to patients. These ventilators typically comprise a source of pressurized oxygen which is fluidly connected to the patient through a conduit or tubing. As each patient may require a different ventilation strategy, modern ventilators can be customized for the particular needs of an individual patient. For example, several different ventilator modes or settings have been created to provide better ventilation for patients in various different scenarios.

### Methods and Systems for Adaptive Adjustment of Ventilator Settings

This disclosure describes systems and methods for adaptively adjusting one or more ventilation settings during ventilation of a patient. The disclosure describes a novel respiratory parameter estimation algorithm that may be used to deliver a target airway pressure calculated based on the estimated respiratory parameters.

In part, this disclosure describes a method for ventilating a patient with a ventilator. The method includes:

a) receiving a plurality of ventilator parameters;

b) estimating a net value of patient muscle effort based on the received ventilator parameters, wherein the net value of patient muscle effort is estimated using a parameter estimate vector update equation to solve for a recursive least squares gain value representing the estimated net value of patient effort at a time instance by subtracting a squared gain value for a previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the saved gain value for the previous time instance multiplied by the regression parameter vector at the time instance from the gain value for the previous time instance; and

c) calculating target inspiratory pressure based on the estimated net value of patient effort.

Yet another aspect of this disclosure describes a ventilator system that includes: a pressure generating system; a ventilation tubing system; one or more sensors; a support module; and parameter identification module. The pressure generating system is adapted to generate a flow of breathing gas. The ventilation tubing system includes a patient interface for connecting the pressure generating system to a patient. The one or more sensors are operatively coupled to at least one of the pressure generating system, the patient, and the ventilation tubing system. The one or more sensors generate output indicative of the inspiration flow. The adaptive calculation module performs quantification of patient muscle effort by establishing a respiratory predictive model of the ventilator based on an equation of motion and received raw measurement parameter data or estimated ventilator parameters, extracting an estimated net value of patient muscle effort by solving for a recursive least squares gain value representing the estimated net value of patient muscle effort at a time instance by subtracting a squared gain value for a previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from the gain value for the previous time instance. The support module optimally adjusts a target inspiratory pressure based on the estimated net value of patient muscle effort or one or more other respiratory parameters derived based on the estimated net value of patient muscle effort.

The disclosure further describes a computer-readable medium having computer-executable instructions for performing a method for ventilating a patient with a ventilator. The method includes:

a) receiving a plurality of ventilator parameters;

b) estimating a net value of patient muscle effort based on the received ventilator parameters, wherein the net value of patient muscle effort is estimated using a parameter estimate vector update equation to solve for a recursive least squares gain value representing the estimated net value of patient muscle effort at a time instance by subtracting a squared gain value for a previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from the gain value for the previous time instance;

c) calculating target inspiratory pressure based on the estimated net value of patient muscle effort; and

d) sending the target inspiratory pressure to a ventilator component to optimally adjust a delivered target airway pressure based on the target inspiratory pressure.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments of systems and methods described below and are not meant to limit the scope of the invention in any manner, which scope shall be based on the claims appended hereto.

FIG. 1 illustrates an embodiment of a ventilator.

FIG. 2 illustrates an embodiment of a method for ventilating a patient on a ventilator during based on an estimated patient muscle effort.

FIG. 3 illustrates an embodiment of method for ventilating a patient on a ventilator based on a desired treatment metric range during an optimized pressure support breath type.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Various embodiments of the present disclosure provide systems and methods for estimating a net value of patient muscle effort based on one or more respiratory parameters. Patient muscle effort may be defined as a physiologic respiratory muscle effort (working against the elasticity of chest wall and lung). In some embodiments, the estimated net value of patient muscle effort may be derived from measured input signals, such as measured pressure and measured flow, and used to estimate the respiratory parameters. The respiratory parameters may include, but are not limited to, lung compliance, patient resistance, and/or tubing compliance. In some embodiments, the estimated net value of patient muscle effort may be used as a substitute for patient effort in subsequent ventilator actions. In other embodiments, methods of the present disclosure allow the respiratory parameters to be continuously provided. In this manner, a net value for patient muscle effort may be determined, as well as an optimal target inspiratory pressure to be delivered to a patient.

In some embodiments of the present disclosure, a relationship between measurable pressure, measurable flow and an unknown patient muscle effort is exploited to estimate a net value of patient muscle effort and an optimal target inspiratory pressure. For instance, the estimate of patient muscle effort may be used recursively to derive a more accurate target inspiratory pressure during succeeding calculation periods. Thus, through use of recursion, the accuracy of target inspiratory pressure may be continuously improved. In one embodiment, ongoing inputs of measured pressure and measured flow are plugged into an adaptive algorithm to estimate patient muscle effort and, as needed, one or more respiratory parameters.

In some cases, an estimated net value of patient muscle effort signal may be used to trigger a ventilation cycle. It is desirable to synchronize the onset and end of a ventilation cycle to effort a patient may be making to breathe on their own (i.e., patient effort). For example, it is desirable to have an accurate ventilator trigger, whereby the ventilator initiates a breath as soon as the patient attempts to inhale. Some ventilators use a pressure trigger which senses a change in ventilation circuit pressure caused by the patient attempting to inhale, while other ventilators use a flow trigger which senses a change in flow caused by the patient attempting to inhale. In either case, delays between the patient's effort and the ventilator response can occur due to a variety of reasons. For example, a leak in the ventilation circuit may allow air to enter the circuit when the patient inhales. Since the entirety of the patient breath is not measured by a ventilator flow sensor, and the ventilator may be monitoring a change in flow to detect an inhalation (flow trigger), the ventilator may be delayed in initiating the breath. Some embodiments of the present disclosure facilitate improved synchronization through providing a reasonably accurate estimate of patient effort that may be used either alone or in relation to other signals to trigger the onset and end of a ventilation cycle. Use of such values may allow a ventilation system to more accurately synchronize mechanical ventilation with the efforts being made by a patient to breathe on their own. Of note, the estimated net value of patient muscle effort and the target inspiratory pressure may be inputs to the same model, and may be calculated using interdependent equations derived from that same model. In one or more embodiments of the present disclosure, the estimated patient muscle effort may be additionally used in relation to controlling pressure support ventilation of a patient. Such pressure support ventilation operates to deliver a gas to a patient in proportion to the patient muscle effort to receive such gas. In various embodiments of the present disclosure, the estimated patient muscle effort and/or respiratory parameters may be used to drive a graphical display that may be used by a clinician for patient monitoring and/or diagnostic purposes.

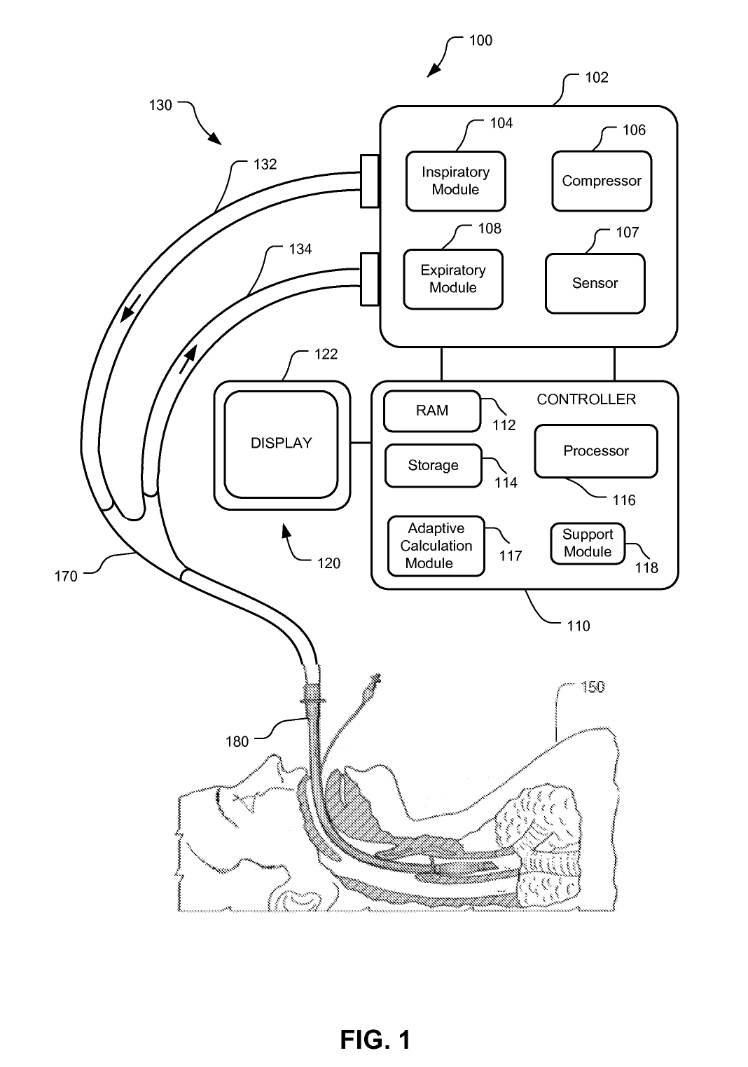

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, pneumatic system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 and the expiratory module 108 to provide a gas source for ventilatory support via inspiratory limb 132.

The inspiratory module 104 is configured to deliver gases to the patient 150 according to prescribed ventilatory settings. In some embodiments, inspiratory module 104 is configured to provide ventilation according to various breath types, e.g., via volume-control, pressure-control, OPA, or via any other suitable breath types.

The expiratory module 108 is configured to release gases from the patient's lungs according to prescribed ventilatory settings. Specifically, expiratory module 108 is associated with and/or controls an expiratory valve for releasing gases from the patient 150.

The ventilator 100 may also include one or more sensors 107 communicatively coupled to ventilator 100. The sensors 107 may be located in the pneumatic system 102, ventilation tubing system 130, and/or on the patient 150. The embodiment of FIG. 1 illustrates a sensor 107 in pneumatic system 102.

Sensors 107 may communicate with various components of ventilator 100, e.g., pneumatic system 102, other sensors 107, processor 116, adaptive calculation module 117, support module 118, and any other suitable components and/or modules. In one embodiment, sensors 107 generate output and send this output to pneumatic system 102, other sensors 107, processor 116, adaptive calculation module 117, support module 118 and any other suitable components and/or modules. Sensors 107 may employ any suitable sensory or derivative technique for monitoring one or more patient parameters or ventilator parameters associated with the ventilation of a patient 150. Sensors 107 may detect changes in patient parameters indicative of patient triggering, for example. Sensors 107 may be placed in any suitable location, e.g., within the ventilatory circuitry or other devices communicatively coupled to the ventilator 100. Further, sensors 107 may be placed in any suitable internal location, such as, within the ventilatory circuitry or within components or modules of ventilator 100. For example, sensors 107 may be coupled to the inspiratory and/or expiratory modules for detecting changes in, for example, circuit pressure and/or flow. In other examples, sensors 107 may be affixed to the ventilatory tubing or may be embedded in the tubing itself. According to some embodiments, sensors 107 may be provided at or near the lungs (or diaphragm) for detecting a pressure in the lungs. Additionally or alternatively, sensors 107 may be affixed or embedded in or near wye-fitting 170 and/or patient interface 180. Indeed, any sensory device useful for monitoring changes in measurable parameters during ventilatory treatment may be employed in accordance with embodiments described herein.

As should be appreciated, with reference to the Equation of Motion, ventilatory parameters are highly interrelated and, according to embodiments, may be either directly or indirectly monitored. That is, parameters may be directly monitored by one or more sensors 107, as described above, or may be indirectly monitored or estimated/calculated using a model, such as a model derived from the Equation of Motion (e.g., Target Airway Pressure(t)=Eₚ∫Qₚdt+QₚRₚ−Patient Effort(t)).

The pneumatic system 102 may include a variety of other components, including mixing modules, valves, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.).

In one embodiment, the operator interface 120 of the ventilator 100 includes a display 122 communicatively coupled to ventilator 100. Display 122 may be included as part of ventilator 100 to allow for interaction with a user including, but not limited to, receiving user commands and/or displaying data relevant to ventilator operation. Display 122 provides various input screens, for receiving clinician input, and various display screens, for presenting useful information to the clinician. In one embodiment, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122. Display 122 may also provide useful information in the form of various ventilatory data regarding the physical condition of a patient 150. The useful information may be derived by the ventilator 100, based on data collected by a processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100. In some embodiments, ventilator 100 may direct display 122 to display information provided by adaptive calculation module 117. Such information may include, but is not limited to, an estimated net value of patient muscle effort as is more fully discussed below.

Controller 110 may include memory 112, one or more processors 116, storage 114, and/or other components of the type commonly found in command and control computing devices. Controller 110 may further include an adaptive calculation module 117, and a support module 118, configured to deliver gases to the patient 150 according to prescribed breath types as illustrated in FIG. 1. In alternative embodiments, adaptive calculation module 117 and the support module 118 may be located in other components of the ventilator 100, such as the processor 116, pneumatic system 102, and/or a separate computing device in communication with the ventilator 100.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

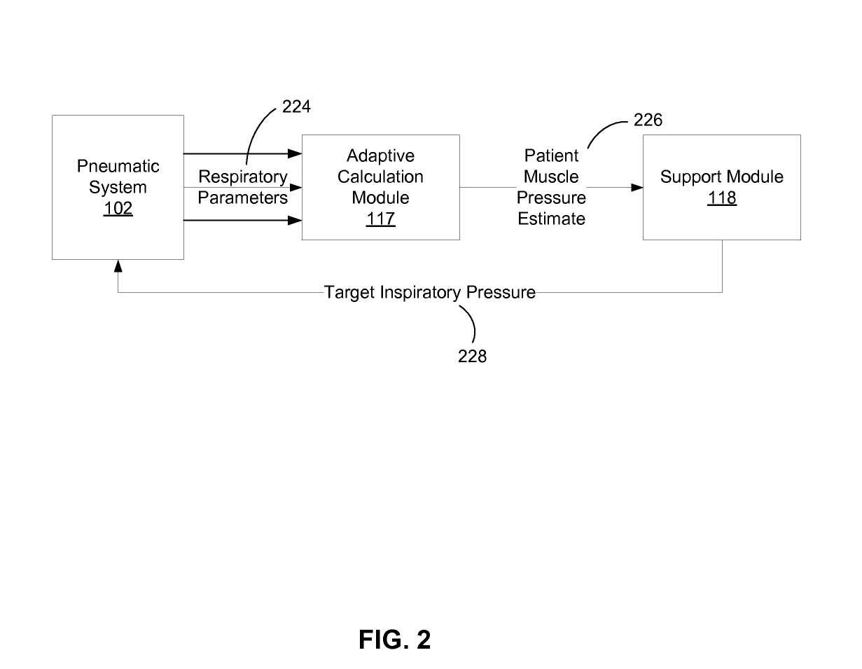

Initiation and execution of an optimal support breath type has two operation prerequisites: (1) estimation of a patient's respiratory muscle effort; and (2) maintaining a target inspiratory pressure 150 during inspiration. Thus, to provide an optimal support breath type, calculation of an estimated net value of patient effort Pₘᵤₛ may first be performed (e.g., by the adaptive calculation module 117). The estimated net value of patient effort as used herein represents the amount of patient effort exerted by a patient's respiratory muscle within a control cycle k (e.g., for a last delivered breath). The estimated net value of patient effort is calculated based on the equation of motion and estimated patient parameters. For instance, the adaptive calculation module 117 receives operator input indicative of, receives measurements indicative of, or estimates one or more patient-ventilator characteristics, or respiratory parameters 224 as illustrated in FIG. 2. The respiratory parameters 224 represent estimates or values of parameters of interest associated with static or dynamic properties or attributes of the ventilated patient system. These values or estimates may be used to adjust ventilator parameters in real time such that closed loop dynamics quickly approach those of a specified system, as described further below. In some embodiments, the adaptive calculation module 117 receives respiratory parameters 224 in the form of raw measurement data or estimates from various sensors that may be part of the patient-ventilator system, including but not limited to physiological sensors, pressure sensors, flow sensors and the like. For instance, inspiratory flow Qᵢₙₛₚ, expiratory flow Qₑₓₕ, inspiratory pressure Pᵢₙₛₚ, and/or expiratory pressure Pₑₓₕ may be measured by flow sensors and/or pressure transducers. Inspiratory limb resistance Rᵢ, expiratory limb resistance Rₑ, tubing compliance (including inspiratory and expiratory limb compliance) C_{RS}, and flow rate in the tube Qₜ, may be estimated using known techniques. Generally throughout, units for pressure may be cm H₂O, units for flow rate may be liters/sec, units for compliance may be liters/cm H₂O, units for resistance may be cm H₂O/liters/sec, and units for volume may be liters.

Various equations may be used to describe the operation of ventilator system 100. For instance, the ventilator 100 may estimate patient parameters based on the measurements directly or indirectly related to monitored patient parameters. In some embodiments, the estimated patient parameters include lung compliance (inverse of elastance) and/or lung/airway resistance. For instance in a healthy lung Pₘᵤₛ is dissipated to overcome the resistance (R_{RS} and elastance

$$\frac{1}{{C}_{RS}}$$

of the respiratory system (inertia is assumed to be negligible), as expressed in the following equation:

$$- {P}_{mus} = {Q}_{L} \times {R}_{RS} + \frac{1}{{C}_{RS}} \times {V}_{L}$$

where Q_{L} denotes the lung flow rate and V_{L} denotes the lung volume.

In further embodiments, the estimated lung compliance, lung elastance and/or lung/airway resistance are estimated based on monitored flow and/or the equation of motion. The estimated patient parameters may be estimated by any processor found in the ventilator 100. In some embodiments, the estimated patient parameters are calculated by the controller 110, the pneumatic system 102, and/or a separate computing device operatively connected to the ventilator 100.

During assisted medical ventilation, there is an interaction between the patient and ventilator. This interaction mainly depends on the response of the ventilator (i.e., P_{aw}) to the patient's effort (i.e., Pₘᵤₛ). Specifically, the pressure provided by the ventilator airway pressure P_{aw} is added to the muscle pressure Pₘᵤₛ, i.e. in medically ventilated patients, the driving pressure for inspiratory flow is the sum of −Pₘᵤₛ and P_{aw}. A good response of the ventilator to the patient's effort results in an appropriate level of ventilator support and a synchrony between the patient and ventilator, which means the ventilator-assisted breathing coincides with the patient's intrinsic breathing pattern in both inhalation phase and exhalation phase.

According to the equation of motion, Pₘᵤₛ is dissipated to overcome the resistance (R_{RS}) and elastance

$$( \frac{1}{{C}_{RS}} )$$

of the respiratory system, as expressed in the following airway pressure equation:

$${P}_{aw} - {P}_{mus} = {Q}_{L} \times {R}_{RS} + \frac{1}{{C}_{RS}} \times {V}_{L}$$

Using the above equation, the adaptive calculation module 117 may then manipulate various received signals in such a way that they meet the requirements of the next stage for further processing (e.g., by provide an estimated net value of patient muscle effort). Assessment of appropriate level of ventilatory support and patient-ventilator synchrony greatly relies on good estimate of a variety of unknown and time-varying respiratory parameters, including the resistance and compliance of respiratory system, and the patient's muscle pressure (i.e. a patient's own breathing effort). Initially, the adaptive calculation module 117 establishes a respiratory predictive model of the ventilated patient system based on the equation of motion and one or more functions that approximate clinically-observed, patient-generated muscle pressures. The respiratory predictive model may be reestablished, updated and/or optimized. An estimated net value of muscle effort value may be extracted from the model. In some instances, a recursive least squares (RLS) based parameter adaptation algorithm is used by the adaptive calculation module 117 to estimate the respiratory parameters using the measured patient's information. According to one embodiment, the adaptive calculation module 117 may first transform the raw sensor measurements and estimates into data in a form useable by the support module 118. For example, pressure and flow sensor data may be digitized and input into the adaptive calculation module 117 to calculate a patient airway pressure using one of the following equations:

P_{aw}(k)=Pᵢₙₛₚ(k)−Qᵢₙₛₚ(k)×Rᵢ(k)

or

P_{aw}(k)=Pₑₓₕ(k)−Qₑₓₕ(k)×Rₑ(k)

where k is a control cycle index. Patient lung resistance R_{L} and compliance C_{L}, chest wall reistance R_{CW} and compliance C_{CW} and patient muscle pressure Pₘᵤₛ may also be identified using the patient airway pressure algorithm. For instance, one or more parameters may be derived from the equations:

$${P}_{aw} ( k ) - {P}_{pl} ( k ) = {Q}_{L} ( k ) \times {R}_{L} ( k ) + \frac{1}{{C}_{L} ( k )} \times \sum {Q}_{L} ( k )$$

$$and$$

$${P}_{pl} ( k ) - {P}_{mus} ( k ) = {Q}_{L} ( k ) \times {R}_{CW} ( k ) + \frac{1}{{C}_{CW} ( k )} \times \sum {Q}_{L} ( k )$$

where Pₚₗ denotes pleural pressure at control cycle k. Adding the above equations obtains the following equation:

$${P}_{aw} ( k ) = {Q}_{L} ( k ) \times {R}_{RS} ( k ) + \frac{1}{{C}_{RS} ( k )} \times {V}_{L} ( k ) + {P}_{mus} ( k )$$

$$where$$

$${R}_{RS} ( k ) = {R}_{L} ( k ) + {R}_{CW} ( k )$$

$$and$$

$$\frac{1}{{C}_{RS} ( k )} = \frac{1}{{C}_{L} ( k )} + \frac{1}{{C}_{CW} ( k )} ,$$

which represent the resistance and elastance of the respiratory system, and V_{L}(k) denotes lung volume at control cycle k.

From the forgoing, it is possible to derive a parameterized output model in a linear regression form. For instance, a first step in defining a parameterized linear regression output model includes defining an unknown parameter vector. In embodiments of the disclosure, the above patient airway pressure equation may be rewritten as a parameterized vector:

$$\begin{matrix}{P}_{aw} ( k ) = [ {R}_{RS} ( k ) \frac{1}{{C}_{RS} ( k )} {P}_{mus} ( k ) ] [ \begin{matrix}{Q}_{L} ( k ) \\ {V}_{L} ( k ) \\ 1\end{matrix} ] \\ = {θ}^{T} ( k ) ϕ ( k ) \\ = {ϕ}^{T} ( k ) θ ( k )\end{matrix}$$

$$where$$

$${θ}^{T} ( k ) = [ {R}_{RS} ( k ) \frac{1}{{C}_{RS} ( k )} {P}_{mus} ( k ) ]$$

is the respiratory parameter vector to be estimated, and

$${ϕ}^{T} ( k ) = [ \begin{matrix}{Q}_{L} ( k ) \\ {V}_{L} ( k ) \\ 1\end{matrix} ]$$

is the regression parameter vector, which may be directly measured or indirectly calculated.

Based on the above parameterized vector equation, estimated patient airway pressure may be defined as:

$$\begin{matrix} ( k ) = {\hat{θ}}^{T} ( k ) ϕ ( k ) \\ = {ϕ}^{T} ( k ) \hat{θ} ( k ) \\ = \end{matrix}$$

$$where {\hat{θ}}^{T} ( k ) = [ ( k ) \frac{1}{ ( k )} ( k ) ]$$

is the estimated respiratory parameter vector.

The adaptive calculation module 117 may perform continuous quantification of respiratory muscle effort of a patient with reference to the above estimated respiratory parameter vector including one or more parameters of the ventilated patient system. As a result, the estimated patient muscle effort [symbol] (k) may be derived and may asymptotically converge to Pₘᵤₛ (k) as k increases. Thus, the parameter identification algorithm provides an optimized solution which solves the estimated parameter vector \hat{θ}(k) while minimizing the following cost function:

$$G ( \hat{θ} ( k ) ) = \frac{1}{2} {\sum }_{j = 0}^{k} {[ {P}_{aw} ( j ) - {ϕ}^{T} ( j ) \hat{θ} ( k ) ]}^{2}$$

where \hat{θ}(k) may be determined by taking partial derivatives for all entries of (k) of \hat{θ}(k) and setting the results to zero:

$$\frac{d G ( \hat{θ} ( k ) )}{d \hat{θ} ( k )} = 0$$

The regression model may then be applied directly in a recursive least squares formulation to solve for the estimated respiratory parameter vector \hat{θ}^{T}(k). For instance, using the estimated respiratory parameter vector, an adaptive algorithm may then be used to determine \hat{θ}^{T}(k) at control cycle k such that \hat{θ}^{T}(k)→θ^{T}(k) as k→∞. OT (k) may then be used to solve for the estimates of Pₘᵤₛ. Specifically, upon solving for the vector

$${\hat{θ}}^{T} ( k ) = [ ( k ) \frac{1}{ ( k )} ( k ) ]$$

each element of the vector is known. Thus, [symbol] (k), which is one element of the vector \hat{θ}^{T} (k), is automatically available when the vector \hat{θ}^{T} (k) is calculated. The estimated respiratory parameter vector \hat{θ}(k) and the regression parameter vector φ(k) may then be fed to the adaptive calculation module 117 which is configured to determine a desired gain value which may then be used to determine a target inspiratory pressure. For instance, the estimated net value of patient muscle effort [symbol] may be derived using the below parameter estimate vector update equation. At a high-level, the computationally efficient adaptive parameter identification algorithm for determining patient respiratory effort as a gain value in accordance with one embodiment of the present disclosure is generally described using the following parameter estimate vector update equations:

$${\hat{θ}}^{T} ( k ) = {\hat{θ}}^{T} ( k - 1 ) + F ( k ) ϕ ( k ) {e}^{°} ( k )$$

$${e}^{°} ( k ) = {P}_{aw} ( k ) - {ϕ}^{T} ( k ) \hat{θ} ( k - 1 )$$

$$F ( k ) = F ( k - 1 ) - \frac{F ( k - 1 ) ϕ ( k ) {ϕ}^{T} ( k ) F ( k - 1 )}{1 + {ϕ}^{T} ( k ) F ( k - 1 ) ϕ ( k )}$$

where F(k)=F^{T}(k)>0 is the RLS gain at the control cycle k.

Thus, an estimated net value of patient muscle pressure [symbol] (designated patient muscle pressure estimate 226 in FIG. 2) may be derived by the adaptive calculation module 117 using the parameter estimate vector update equation, as described above. Specifically, the parameter estimate vector update equation may solve for a recursive least squares gain value representing the net value of patient effort at a time instance based on a squared gain value for a previous time instance by subtracting a squared gain value for the previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from a gain value for the previous time instance. The end result of the above calculation will provide an estimated net value of patient muscle pressure [symbol] . In some embodiments, the estimated net value of patient effort [symbol] may be defined as the estimation of a net value of the dynamic pressure developed by a patient's respiratory muscle.

In some embodiments, the parameter estimate vector update equation may be modified by introducing a forgetting factor 0<μ<1, such that the update equation becomes:

$$F ( k ) = \frac{1}{μ} [ F ( k - 1 ) - \frac{F ( k - 1 ) ϕ ( k ) {ϕ}^{T} ( k ) F ( k - 1 )}{μ + {ϕ}^{T} ( k ) F ( k - 1 ) ϕ ( k )} ]$$

In such instances, the closer μ is to 1, the less responsive the adaptive parameter estimation will be to parameter variations.

The adaptive calculation module 117 may calculate an estimated net value of patient effort [symbol] for every delivered breath or every breath delivered after a predetermined amount of time or after a predetermined event. In some instances, quantification is performed online (i.e., during ventilator operation). In some embodiments, the estimated net value of [symbol] value extracted from the update equation may be compensated for time delays introduced by the ventilator's measurement system and/or the indirect indication of muscular activity by surrogate phenomena (e.g., pressure).

The estimated net value of patient muscle effort [symbol] may include a solitary value. Accordingly, the estimated patient effort may be, for instance, 5 cm of H₂O, 6 cm of H₂O, 7 cm of H₂O, 8 cm of H₂O, 9 cm of H₂O, or 10 cm of H₂O. The provided examples are not limiting.

Upon calculation, the estimated net value of patient muscle effort [symbol] may be provided to one or more other ventilator components to configure the ventilation system based on the estimated net value of patient muscle effort to provide an optimal support breath type. As illustrated in FIG. 2, in some embodiments, the support module 118 receives the estimated net value of muscle effort value from the adaptive calculation module 117 (e.g., as patient muscle pressure estimate 226). In other embodiments, the support module 118 may receive the estimated net value of patient effort [symbol] from one or more other ventilator components, such as processor 116, and/or operator interface 120.

To accomplish the second operation prerequisite of determining target inspiratory pressure to be delivered to the patient 150 during inspiration, the support module 118 utilizes the received estimated net value of patient muscle effort to estimate a target inspiratory pressure. For a given control cycle k, with the estimated patient effort [symbol] (k), the support module 118 may optimally adjust the ventilator support level. For instance, using [symbol] , the ventilator support level may be optimized by progressively adjusting the target inspiratory pressure P_{target}. In some embodiments, at the time instant k, the target inspiratory pressure at a next time instant can be adjusted using the following optimization algorithm:

$${P}_{target} ( k + 1 ) = ( k ) \times {Q}_{L} ( k ) + \frac{1}{ ( k )} \times {V}_{L} ( k ) + ( k )$$

where P_{target}(k+1) is the target inspiratory pressure at control cycle k+1.

The target inspiratory pressure P_{target} may be used to command the pressure control. For instance, the support module 118 may send the target inspiratory pressure P_{target} (designated as target inspiratory pressure 228 in FIG. 2) to the appropriate component or components of the ventilator 100, such as the pneumatic system 102, controller 110, and/or processor 116, of the ventilator 100 for adjusting the target inspiratory pressure and/or one or more other ventilator parameters. In some instances, the support module 118 sends the target inspiratory pressure to another ventilator component (e.g., a feedback controller, actuator, or directly to the patient circuit). The receiving component may then send an optimal support breath type to the inspiratory module 104. The optimal support breath type refers to a type of ventilation in which the ventilator 100 acts as an inspiratory amplifier that provides pressure support to the patient. The degree of amplification (the “pressure support setting”) is determined by the support module 118 based on the estimated net value of patient muscle effort received from the adaptive calculation module 117. Various feedback control regimes may be implemented, including pressure, volume, and/or flow regulation. Feedback control may also be predicated on inputs received from the patient, such as pressure variations in the breathing circuit which indicate commencement of inspiration.

In some embodiments, the optimization algorithm utilized by the support module 118 incorporates an internal model of the patient respiratory system in interaction with the ventilator 100 to address the relevant interactive dynamics between the patient 150 and the ventilator 100 as well as model and predict changes in patient's respiratory behavior and therapeutic outcome in response to the ongoing treatment protocol delivered by the ventilator 100. In some embodiments, the internal model for the optimization algorithm incorporates mechanisms for estimating system parameters (respiratory resistance, compliance, and etc.). Additionally, the optimization algorithm utilized by the support module 118 may include features to estimate, model, or predict dynamics related to the functioning and interrelationships between inputs (e.g., pressure support value, SpO₂, oxygen mix, and etc.) and output (generated patient effort over time). In further embodiments, the optimization algorithm includes mechanisms to estimate physiologic-based and/or hardware-based dynamics (transients, delays, and etc.).

In some embodiments, one or more ventilator parameters and their derivatives may be adjusted based information received from the support module 118. The support module 118 sends the target inspiratory pressure command to, for instance, the patient circuit. A treatment module or other ventilator component then causes the ventilator 100 to deliver a target airway pressure based on the calculated patient effort and target inspiratory pressure to the patient during the utilization of the optimal support breath type. Accordingly, the support module 118 may send the target inspiratory pressure and/or instruction for delivering a target airway pressure to at least one of the processor 116, pneumatic system 102, inspiratory module 104 and/or the controller 110. In such embodiments, a ventilator component such as a treatment module may be configured to adjust the percent support setting and the ventilator parameters by utilizing algorithms and optimization programming techniques to provide advisory input and/or automatic adjustments to ventilation parameters (e.g., oxygen percentage) and/or a timed changes in ventilation modality (patient-triggered or ventilator-driven breath delivery) to increase the efficiency and confidence in the predictive nature of the treatment success/failure indices. In some embodiments, the ventilator parameters are adjusted based on treatment optimization algorithm. In other words, the algorithms and optimization programming techniques adjust the percent support setting and the one or more ventilator parameters in an attempt to improve patient treatment (i.e., maintain a current treatment metric within a desired treatment metric range).

In some instances, the estimated patient muscle effort [symbol] may be utilized to determine onset and end of a ventilation cycle. Upon determining at least one of onset and/or end of a ventilation cycle, ventilator support may be synchronized with patient effort. During an exhalation phase, if the negative deflection of [symbol] is greater than a preset triggering sensitivity value Pₛₑₙ, the onset of an inspiration (i.e., the end of a previous expiration) may be indicated. Further, during an inhalation phase, if the positive deflection of [symbol] is greater than Pₛₑₙ, the onset of an expiration (i.e., the end of a current inspiration) may be indicated. Based on at least one of the described indicators, the ventilator may achieve improved synchrony with a patient.

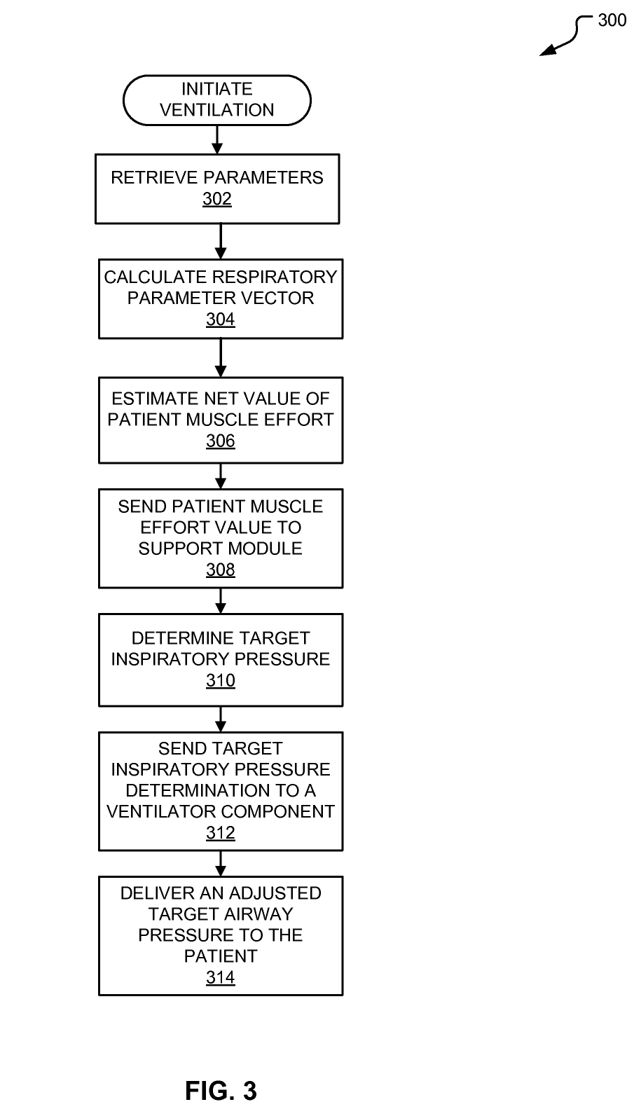

FIG. 3 illustrates an embodiment of a method 300 for ventilating a patient with a ventilator that utilizes an optimal support breath type. The optimal support breath type delivers a target airway pressure calculated based on a target inspiratory pressure value derived from an estimated net value of patient effort. The estimated net value of patient effort allows the ventilator to maintain a desired patient effort by optimally adjusting a ventilator support level. Further, determining a target inspiratory pressure can be utilized to more accurately estimate the onset and end of a ventilation cycle during treatment of a patient on a ventilator.

Method 300 illustrates the method for delivering a breath during an optimal support breath type. Method 300 begins after the initiation of ventilation.

As illustrated, method 300 includes a retrieving operation 302. During the retrieving operation 302, the ventilator retrieves one or more ventilator parameters. In some cases, the pressure may be measured in a tube connecting a ventilator to a person being ventilated. In some cases, the pressure is measured near a gas inlet and/or near a gas outlet. In other cases, the pressure is measured near a junction of the gas inlet with the gas outlet. In various cases, the pressure measurement is a single point pressure measurement, while in other cases the pressure measurement is a multiple point pressure measurement and the measured pressure is a mathematical combination of two or more pressure measurements. Retrieving the inlet flow may include retrieving a measured flow of a single gas, or measuring the flows of two or more gases and aggregating the multiple flow values. Retrieving the outlet flow may include, but is not limited to, measuring the flow of gas at the outlet of the ventilation system. The outlet flow is subtracted from the inlet flow at a particular instance to generate an instantaneous net flow. Any of the above described parameters may also be estimates as described above.

Method 300 also includes a first calculating operation 304. For instance, the net flow and measured pressure for a given instant may be used to calculate an estimated respiratory parameter vector based on the equation:

$$\begin{matrix}{P}_{aw} ( k ) = {θ}^{T} ( k ) ϕ ( k ) \\ = {ϕ}^{T} ( k ) θ ( k ) \\ = [ {R}_{RS} ( k ) \frac{1}{ ( k )} {P}_{mus} ( k ) ] [ \begin{matrix}{Q}_{L} ( k ) \\ {V}_{L} ( k ) \\ 1\end{matrix} ] ,\end{matrix}$$

$$where {θ}^{T} ( k ) = [ {R}_{RS} ( k ) \frac{1}{{C}_{RS} ( k )} {P}_{mus} ( k ) ]$$

is the respiratory parameter vector to be estimated, and

$${ϕ}^{T} ( k ) = [ \begin{matrix}{Q}_{L} ( k ) \\ {V}_{L} ( k ) \\ 1\end{matrix} ]$$

is the regression parameter vector, which may be directly measured or indirectly calculated.

Method 300 also includes an estimating operation 306. For instance, the estimated respiratory parameter vector \hat{θ}^{T} may then be used to estimate a net value for patient muscle effort [symbol] at the given control cycle k. The process of calculating an estimated a patient muscle effort [symbol] may be done using the approach discussed above in relation to FIG. 1. Based on the above parameterized vector equation, estimated patient airway pressure may be defined as:

$$\begin{matrix} ( k ) = {\hat{θ}}^{T} ( k ) ϕ ( k ) \\ = {ϕ}^{T} ( k ) \hat{θ} ( k ) \\ = [ ( k ) \frac{1}{ ( k )} ( k ) ] [ \begin{matrix}{Q}_{L} ( k ) \\ {V}_{L} ( k ) \\ 1\end{matrix} ] ,\end{matrix}$$

$$where {\hat{θ}}^{T} ( k ) = [ ( k ) \frac{1}{ ( k )} ( k ) ]$$

is the estimated respiratory parameter vector.

The adaptive calculation module 117 may perform continuous quantification of respiratory muscle effort of a patient with reference to the above estimated respiratory parameter vector including one or more parameters of the ventilated patient system. As a result, the estimated patient muscle effort [symbol] (k) may be derived and may asymptotically converge to Pₘᵤₛ (k) as k increases. Thus, the parameter identification algorithm provides an optimal solution which solves the estimated parameter vector \hat{θ}(k) while minimizing the following cost function:

$$G ( \hat{θ} ( k ) ) = \frac{1}{2} {\sum }_{j = 0}^{k} {[ {P}_{aw} ( j ) - {ϕ}^{T} ( j ) \hat{θ} ( k ) ]}^{2}$$

where \hat{θ}(k) may be determined by taking partial derivatives for all entries of (k) of \hat{θ}(k) and setting the results to zero:

$$\frac{d G ( \hat{θ} ( k ) )}{d \hat{θ} ( k )} = 0$$

The regression model may then be applied directly in a recursive least squares formulation to solve for the estimated respiratory parameter vector \hat{θ}^{T} (k). For instance, using the estimated respiratory parameter vector, an adaptive algorithm may then be used to determine \hat{θ}^{T}(k) at control cycle k such that \hat{θ}^{T}(k)→θ^{T}(k) as k→∞.

\hat{θ}^{T}(k) may then be used to solve for the estimates of Pₘᵤₛ. Specifically, upon solving for the vector

$${\hat{θ}}^{T} ( k ) = [ ( k ) \frac{1}{ ( k )} ( k ) ]$$

each element of the vector is known. Thus, [symbol] (k), which is one element of the vector \hat{θ}^{T}(k), is automatically available when the vector \hat{θ}^{T}(k) is calculated. The estimated respiratory parameter vector \hat{θ}(k) and the regression parameter vector φ(k) may then be fed to the adaptive calculation module 117 which is configured to determine a desired gain value which may then be used to determine a target inspiratory pressure. For instance, the estimated net value of patient muscle effort [symbol] may be derived using the below parameter estimate vector update equation. At a high-level, the computationally efficient adaptive parameter identification algorithm for determining patient respiratory effort as a gain value in accordance with one embodiment of the present disclosure is generally described using the following parameter estimate vector update equations:

$${\hat{θ}}^{T} ( k ) = {\hat{θ}}^{T} ( k - 1 ) + F ( k ) ϕ ( k ) {e}^{°} ( k )$$

$${e}^{°} ( k ) = {P}_{aw} ( k ) - {ϕ}^{T} ( k ) \hat{θ} ( k - 1 )$$

$$F ( k ) = F ( k - 1 ) - \frac{F ( k - 1 ) ϕ ( k ) {ϕ}^{T} ( k ) F ( k - 1 )}{1 + {ϕ}^{T} ( k ) F ( k - 1 ) ϕ ( k )}$$

where F(k)=F^{T} (k)>0 is the RLS gain at the control cycle k.

Thus, an estimated net value of patient muscle pressure [symbol] (designated patient muscle pressure estimate 226 in FIG. 2) may be derived by the adaptive calculation module 117 using the parameter estimate vector update equation, as described above. Specifically, the parameter estimate vector update equation may solve for a recursive least squares gain value representing the net value of patient effort at a time instance based on a squared gain value for a previous time instance by subtracting a squared gain value for the previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from a gain value for the previous time instance. The end result of the above calculation will provide an estimated net value of patient muscle pressure [symbol] . In some embodiments, the estimated net value of patient effort [symbol] may be defined as the estimation of a net value of the dynamic pressure developed by a patient's respiratory muscle.

Next, method 300 includes a first sending operation 308. During the first sending operation 308, the adaptive parameter module 117 sends estimated patient muscle effort [symbol] (k) to the support module 118 for determining an optimal target inspiratory pressure.

Next, method 300 includes a determining operation 310. During the determining operation 310, the support module 118 uses the received estimated patient muscle effort [symbol] (k) to determine an optimal target inspiratory pressure. As illustrated in FIG. 2, in some embodiments, the support module 118 receives the estimated net value of muscle effort value from the adaptive calculation module 117 (e.g., as patient muscle pressure estimate 226). In other embodiments, the support module 118 may receive the estimated net value of patient effort from one or more other ventilator components, such as processor 116, and/or operator interface 120. To accomplish the second operation prerequisite of determining target inspiratory pressure to be delivered to the patient 150 during inspiration, the support module 118 utilizes the received estimated net value of patient muscle effort to estimate a target inspiratory pressure. For a given control cycle k, with the estimated patient effort [symbol] (k), the support module 118 may optimally adjust the ventilator support level. For instance, using [symbol] , the ventilator support level may be optimized by progressively adjusting the target inspiratory pressure P_{target}. In some embodiments, at the time instant k, the target inspiratory pressure at a next time instant can be adjusted as:

$${P}_{target} ( k + 1 ) = ( k ) \times {Q}_{L} ( k ) + \frac{1}{ ( k )} \times {V}_{L} ( k ) + ( k )$$

where P_{target}(k+¹) is the target inspiratory pressure at control cycle k+1.

Method 300 also includes a second sending operation 312. During the second sending operation 312, the support module 118 sends the determined target inspiratory pressure P_{target} to another ventilator component (e.g., a feedback module). The target inspiratory pressure P_{target} may be used to command the pressure control. For instance, the support module 118 may send the target inspiratory pressure P_{target} (designated as target inspiratory pressure 228 in FIG. 2) to the appropriate component or components of the ventilator 100, such as the pneumatic system 102, controller 110, and/or processor 116, of the ventilator 100 for adjusting the target inspiratory pressure and/or one or more other ventilator parameters. In some instances, the support module 118 sends the target inspiratory pressure to another ventilator component (e.g., a feedback controller, actuator, or directly to the patient circuit). The receiving component may then send an optimal support breath type to the inspiratory module 104. The optimal support breath type refers to a type of ventilation in which the ventilator 100 acts as an inspiratory amplifier that provides pressure support to the patient. The degree of amplification (e.g. a “pressure support setting”) is determined based on the determined target inspiratory pressure P_{target} received from the support module 118. Various feedback control regimes may be implemented, including pressure, volume, and/or flow regulation. Feedback control may also be predicated on inputs received from the patient, such as pressure variations in the breathing circuit which indicate commencement of inspiration.

Method 300 may optionally include a delivery operation 314. During the delivery operation, the ventilator delivers an adjusted target airway pressure to a patient based on the determined target inspiratory pressure P_{target}. The ventilator delivers the adjusted target airway pressure to the patient after the detection of a patient initiated inspiratory trigger. The target airway pressure is delivered after an inspiratory trigger is detected. A patient trigger is calculated based on the at least one monitored parameter, such as inspiration flow. In some embodiments, sensors, such as flow sensors, may detect changes in patient parameters indicative of patient triggering. The target airway pressure delivered by the ventilator during the first delivery operation 212 is an initial target airway pressure calculated by the ventilator based on the initial percent support setting.

In some embodiments, method 300 includes a display operation. The ventilator during the display operation displays any suitable information for display on a ventilator. In one embodiment, the display operation displays at least one of the estimated patient effort, the target inspiratory pressure, the desired treatment metric range, the current treatment metric, the RSBI, the SpO₂, the P100, the tidal volume, the VCO₂, the respiratory rate, the spontaneous I:E volume, the minute volume, the initial percent support setting, and the adjusted percent support setting.

In an additional embodiment, any method for estimating a net value of patient effort may be utilized to obtain and/or maintain a predetermined patient effort based on a calculated flow and/or pressure. This approach provides for gas delivery at a rate and/or pressure to provide and/or maintain an estimated patient effort. Accordingly, this approach provides for increased patient comfort as well as less interference with a patient's own attempts at breathing. According to some embodiments, the inspiratory module 104 and/or the expiratory module 108 may be configured to synchronize ventilation with a spontaneously-breathing, or triggering, patient. That is, the ventilator may be configured to detect patient effort and may initiate a transition from exhalation to inspiration (or from inspiration to exhalation) in response. Triggering refers to the transition from exhalation to inspiration in order to distinguish it from the transition from inspiration to exhalation (referred to as cycling). Ventilation systems, depending on their breath type, may trigger and/or cycle automatically, or in response to a detection of patient effort, or both.

In one embodiment, the adaptive algorithm and/or optimization programming incorporates an internal model of the patient respiratory system in interaction with the ventilator to address the relevant interactive dynamics between the patient and the ventilator as well as model and predict changes in patient's respiratory behavior and therapeutic outcome in response to the ongoing treatment protocol delivered by the ventilator. The control system design of the support module is envisioned to optimize convergence of the control output or desired patient effort. In some embodiments, the internal model for the adaptive algorithm and/or optimization programming incorporates mechanisms for estimating system parameters (respiratory resistance, compliance, and etc.). Additionally, the adaptive algorithm and/or the optimization programming may include features to estimate, model, or predict dynamics related to the functioning and interrelationships between inputs (e.g., percent support, SpO₂, oxygen mix, and etc.) and output (generated patient effort over time). In further embodiments, the adaptive algorithm includes mechanisms to estimate physiologic-based and/or hardware-based dynamics (transients, delays, and etc.).

In some embodiments, a microprocessor-based ventilator that accesses a computer-readable medium having computer-executable instructions for performing the method of ventilating a patient with a medical ventilator is disclosed. This method includes repeatedly performing the steps disclosed in method 300 above and/or as illustrated in FIG. 3.

In some embodiments, the ventilator system includes: means for receiving a plurality of ventilator parameters; means for estimating a net value of patient muscle effort based on the received ventilator parameters, where the net value of patient muscle effort is derived using a parameter estimate vector update equation; and means for calculating target inspiratory pressure using an optimization algorithm based on the estimated net value of patient effort.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for ventilating a patient with a ventilator comprising:
receiving a plurality of ventilator parameters;
estimating a net value of patient muscle effort based on the received ventilator parameters, wherein the net value of patient muscle effort is estimated using a parameter estimate vector update equation to solve for a recursive least squares gain value representing the estimated net value of patient effort at a time instance by subtracting a squared gain value for a previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from the gain value for the previous time instance; and
calculating target inspiratory pressure based on the estimated net value of patient effort.

2. The method of claim 1, wherein receiving a plurality of ventilator parameters includes receiving raw measurement parameter data or parameter estimates from at least one sensor.

3. The method of claim 2, wherein raw measurement parameter data includes at least one of inspiratory flow, expiratory flow, inspiratory pressure, and expiratory pressure.

4. The method of claim 2, wherein parameter estimates include at least one of an estimate of inspiratory limb resistance, expiratory limb resistance, inspiratory limb compliance, expiratory limb and flow rate in a ventilation tube.

5. The method of claim 1, wherein estimating a net value of patient muscle effort includes:
applying a forgetting factor to the parameter estimate vector update equation.

6. The method of claim 1, further comprising calculating the target inspiratory pressure for a next time instance by adding the estimated net value of patient muscle effort at a time instance to the sum of the product of an estimated inverse limb compliance value and lung volume value at the time instance and the product of an estimated resistance value at a time instance and a lung flow rate at the time instance.

7. The method of claim 6, further comprising:
using the target inspiratory pressure to calculate an adjusted target airway pressure by subtracting the calculated target inspiratory pressure at the time instance from the product of a flow rate and a resistance value at the time instance.

8. The method of claim 7, further comprising:
delivering the adjusted target airway pressure to a patient.

9. A ventilator system comprising:
a pressure generating system adapted to generate a flow of breathing gas;
a ventilation tubing system including a patient interface for connecting the pressure generating system to a patient;
one or more sensors operatively coupled to at least one of the pressure generating system, the patient, and the ventilation tubing system, wherein the one or more sensors generate output indicative of an inspiration flow;
an adaptive calculation module adapted to perform quantification of patient muscle by establishing a respiratory predictive model of the ventilator based on an equation of motion and received raw measurement parameter data or estimated ventilator parameters and extracting an estimated net value of patient muscle effort by solving for a recursive least squares gain value representing the estimated net value of patient muscle effort at a time instance by subtracting a squared gain value for a previous time instance multiplied by a regression parameter vector at the time instance and a transpose of the regression parameter vector at the time instance and a transpose of the squared gain value for the previous time instance divided the result by one plus the transpose of the regression parameter vector at the time instance multiplied by the squared gain value for the previous time instance multiplied by the regression parameter vector at the time instance from the gain value for the previous time instance; and
a support module operable to adjust a target inspiratory pressure based on the estimated net value of patient muscle effort or one or more other respiratory parameters derived based on the estimated net value of patient muscle effort.

10. The ventilator system of claim 9, wherein raw measurement parameter data includes at least one of inspiratory flow, expiratory flow, inspiratory pressure, and expiratory pressure.

11. The ventilator system of claim 9, wherein at least one estimated ventilator parameter includes at least one of an estimate of inspiratory limb resistance, expiratory limb resistance, inspiratory limb compliance, expiratory limb and flow rate in a ventilation tube.

12. The ventilator system of claim 9, wherein the adaptive calculation module applies a forgetting factor to the parameter estimate vector update equation to estimate the net value of patient muscle effort.

13. The ventilator system of claim 9, wherein the support module calculates a target inspiratory pressure based at least on the net value of patient muscle effort.

14. The ventilator system of claim 13, wherein the support module calculates the target inspiratory pressure for a next time instance by adding the estimated net value of patient muscle effort at a time instance to the sum of the product of an estimated inverse limb compliance value and lung volume value at the time instance and the product of an estimated resistance value at the time instance and a lung flow rate at the time instance.

15. The ventilator system of claim 14, wherein the support module uses the target inspiratory pressure to calculate an adjusted target airway pressure by subtracting the calculated target inspiratory pressure at the time instance from the product of a flow rate and a resistance value at the time instance.

16. The ventilator system of claim 15, wherein the support module sends the calculated target inspiratory pressure to at least one ventilator component for delivery of the adjusted target airway pressure to a patient.

17. The ventilator system of claim 15, further comprising a display that displays at least one of the net value of patient muscle effort or an initial support setting based on the calculated target inspiratory pressure.

18. A computer-readable medium having computer-executable instructions for performing a method of ventilating a patient with a ventilator, the method comprising:
receiving a plurality of ventilator parameters;
estimating a net value of patient muscle effort based on the received ventilator parameters, wherein the net value of patient muscle effort is estimated using a parameter estimate vector update equation to solve for a recursive least squares gain value representing the estimated net value of patient muscle effort at a time instance by subtracting a squared gain value for a previous time instance multiplied by a regression parameter vector at the time instance and a transpose of the regression parameter vector at the time instance divided the result by one plus a gain value for the previous time instance multiplied by the transpose of the regression parameter vector at the time instance multiplied by the regression parameter vector at the time instance from the gain value for the previous time instance;
calculating target inspiratory pressure based on the estimated net value of patient muscle effort; and
sending the target inspiratory pressure to a ventilator component to optimally adjust a delivered target airway pressure based on the target inspiratory pressure.

19. The ventilator system of claim 18, further comprising calculating the target inspiratory pressure for a next time instance by adding the estimated net value of patient muscle effort at the time instance to the sum of the product of an estimated inverse limb compliance value and lung volume value at the time instance and the product of an estimated resistance value at the time instance and a lung flow rate at the time instance.

20. The ventilator system of claim 18, further comprising:
using the target inspiratory pressure to calculate an adjusted target airway pressure by subtracting the calculated target inspiratory pressure at the time instance from the product of a flow rate and a resistance value at the time instance.
